(19) United States
(12) Patent Application Publication
(10) Pub. No.: US 2026/0331235 A1
(43) Pub. Date: Oct. 22, 2026

(54) METHOD AND SYSTEM FOR AUTOMATING PATIENT-SPECIFIC SPECIMEN COLLECTION

(71) Applicant: AT SOLUTIONS CORP, Siheung-si (KR)

(72) Inventor: Jeong Hyun MOON, Siheung-si (KR)

(73) Assignee: AT SOLUTIONS CORP, Siheung-si (KR)

(21) Appl. No.: 19/704,426

(22) Filed: Jun. 11, 2026

Related U.S. Application Data
(63) Continuation of application No. PCT/KR2023/020312, filed on Dec. 11, 2023.

(51) Int. Cl. G16H 10/40; A61B 5/15; A61B 50/33; G16H 10/60

(52) U.S. Cl. CPC G16H 10/40; A61B 5/150763; A61B 50/33; G16H 10/60

(57) ABSTRACT

A patient-specific specimen collection automation system according to an embodiment of the present disclosure includes a management server including specimen information required for respective patients, a tray supply device configured to supply, to a tray, blood collection tubes labeled to correspond to the specimen information, and a tray transport device configured to transport the tray. The tray supply device includes a supply device communication unit configured to communicate with the management server, a blood collection tube supply unit configured to supply the blood collection tubes labeled to correspond to the specimen information, and a tray supply unit configured to classify the labeled blood collection tubes for respective patients into the tray, the tray including identification information. The tray transport device includes a transport device communication unit configured to communicate with the management server, a tray holder configured to receive the tray supplied from the tray supply device, a tray identification unit configured to obtain the identification information of the tray, and an output unit configured to indicate trays matched to respective patients.

### TECHNICAL FIELD

The present disclosure relates to a method and system for automating patient-specific specimen collection. Specifically, the present invention relates to a method and system for managing specimen information required for each patient through a central management server and automating patient-specific specimen collection using a tray supply device and a tray transport device.

### BACKGROUND

Various tests are performed according to their purposes by collecting, as specimens, blood, urine, stool, sputum, cells, puncture fluid, and the like. The specimen type differs for each patient according to the purpose of the test. In addition, even for the same specimen type, a plurality of specimen collection tools may be required according to the purpose of the test. For example, a plurality of blood collection tubes may be required for each patient according to the purpose of the test.

Conventionally, workers employed at a hospital manually prepared specimen collection tools required for each patient. There was a problem in that a large amount of time was required to manually prepare the specimen collection tools required for each patient. Accordingly, recently, equipment has been provided that automatically labels blood collection tubes by matching the blood collection tubes with a patient's specimen information. However, as specimen collection tools, there are many tools other than blood collection tubes, and a large amount of time is still required to classify such specimen collection tools for each patient.

In addition, when one or more specimen collection tools are omitted in the process of manually preparing specimen collection tools and are therefore not taken to a patient, a problem occurs in that the corresponding specimen collection is omitted. A large amount of time is required to confirm the omitted specimen collection, prepare the omitted specimen collection tool again, and collect the specimen.

In addition, in the process of manually checking specimen collection tools one by one, a problem sometimes occurs in that a specimen of another patient is received in a prepared specimen collection tool. When a specimen of another patient is received in a specimen collection tool, a problem occurs in that the patient's condition may be misdiagnosed, and a large amount of time is required to correct the misdiagnosis.

### SUMMARY

### Problem

An object of the present disclosure is to easily classify specimen collection tools required for respective patients, such as blood collection tubes and urine containers, thereby reducing the time required to prepare and classify specimen collection tools for respective patients.

Another object of the present disclosure is to prevent medical errors by automatically confirming whether specimen collection tools are matched to respective patients.

Another object of the present disclosure is to prevent omissions in specimen collection by centrally managing records of completed specimen collections among specimen information required for respective patients.

### Solution

A patient-specific specimen collection automation system according to an embodiment of the present disclosure includes a management server including specimen information required for respective patients, a tray supply device configured to supply, to a tray, blood collection tubes labeled to correspond to the specimen information, and a tray transport device configured to transport the tray. The tray supply device includes a supply device communication unit configured to communicate with the management server, a blood collection tube supply unit configured to supply the blood collection tubes labeled to correspond to the specimen information, and a tray supply unit configured to classify the labeled blood collection tubes for respective patients into the tray, the tray including identification information. The tray transport device includes a transport device communication unit configured to communicate with the management server, a tray holder configured to receive the tray supplied from the tray supply device, a tray identification unit configured to obtain the identification information of the tray, and an output unit configured to indicate trays matched to respective patients.

In addition, the tray supply unit includes a tray moving unit configured to move the tray, and a tray identification unit configured to confirm the identification information indicated on the tray.

In addition, the tray supply device further includes a label sheet supply unit configured to supply, to the tray, a label sheet corresponding to the specimen information.

In addition, the tray supply device classifies at least one of blood collection tubes labeled for respective patients and label sheets into the identified tray, and transmits identified tray information for respective patients to the management server.

In addition, the tray transport device further includes a label identification unit configured to identify label information.

In addition, the tray transport device identifies label information of a specimen collection tool included in the identified tray, and transmits, to the management server, specimen collection completion information for respective patients corresponding to the identified label information.

A patient-specific specimen collection automation method according to an embodiment of the present disclosure includes inputting specimen information required for respective patients, classifying blood collection tubes labeled to correspond to the specimen information into an identified tray, transporting the identified tray, and identifying trays matched to respective patients.

In addition, the classifying of the labeled blood collection tubes further includes classifying a label sheet corresponding to the specimen information into the identified tray.

In addition, the patient-specific specimen collection automation method further includes identifying label information of a specimen collection tool included in the identified tray, and inputting specimen collection completion information corresponding to the identified label information.

In addition, the identifying of the label information further includes confirming whether the identified label information matches input patient information, and inputting specimen collection completion information corresponding to the identified label information.

A tray supply device according to an embodiment of the present disclosure includes a supply device communication unit configured to communicate with a management server including specimen information required for respective patients, a blood collection tube supply unit configured to label and supply introduced blood collection tubes to correspond to the specimen information required for respective patients, and a tray supply unit configured to classify the blood collection tubes supplied from the blood collection tube supply unit into trays for respective patients. The tray supply unit includes a tray moving unit configured to move a tray, and a tray identification unit configured to confirm identification information indicated on the tray.

In addition, the tray supply device transmits, through the supply device communication unit, tray identification information matched to respective patients to the management server or a tray transport device.

In addition, the tray supply device further includes a label sheet supply unit configured to supply, to the tray, a label sheet corresponding to the specimen information.

In addition, the blood collection tube supply unit includes a blood collection tube inlet hole into which blood collection tubes are introduced, an inlet hole partition configured to partition the blood collection tube inlet hole along one direction, a roller unit provided below the blood collection tube inlet hole, and a blood collection tube discharge hole provided below the roller unit and configured to discharge the blood collection tubes. The roller unit includes a roller unit body provided along the one direction, a roller unit main groove provided in the roller unit body along the one direction and into which a blood collection tube is introduced, and a roller unit sub-groove crossing obliquely along a side surface of the roller unit body.

In addition, the tray supply unit includes a first tray loading unit in which trays filled with blood collection tubes for respective patients are stacked, and a second tray loading unit in which empty trays are stacked and which supplies the empty trays to the tray moving unit.

In addition, the tray moving unit includes a tray guide configured to surround side surfaces of the tray, a guide driving unit configured to move the tray guide along the first tray loading unit, the second tray loading unit, and the tray identification unit, and a vertical driving unit provided below the first tray loading unit and configured to move, by a vertical moving component, a tray filled with blood collection tubes for respective patients to the first tray loading unit.

In addition, the tray has a stepped structure defining an upper portion and a lower portion, and an upper perimeter is larger than a lower perimeter.

In addition, the first tray loading unit includes a first support portion rotatably connected to a rotation shaft and configured to support the tray by engaging with the stepped structure of the tray, a first guide groove configured to limit a rotation angle of the first support portion, and a first sensor unit configured to detect rotation of the first support portion.

In addition, the second tray loading unit includes a second support portion rotatably connected to a rotation shaft and configured to support the tray by engaging with the stepped structure of the tray, a driving motor configured to rotationally drive the second support portion, and a second sensor unit configured to detect rotation of the second support portion.

In addition, the second tray loading unit includes two sets arranged vertically, each set including the second support portion (2341), the driving motor (2346), and the second sensor unit (2345).

A tray transport device according to an embodiment of the present disclosure transports trays loaded with specimen collection tools for respective patients, and includes a drive unit including a driving device for movement, a tray holder including partitioned spaces configured to receive the trays, a tray identification unit provided in the partitioned spaces and configured to identify identification information of the trays, a transport device communication unit configured to communicate with a management server including specimen information required for respective patients and identification information of trays matched to respective patients, an input unit configured to receive patient information, and an output unit configured to indicate a tray matched to the input patient information.

In addition, the output unit is an LED provided in each partitioned space of the tray holder and configured to indicate a section in which a tray matched to the input patient information is received.

In addition, the tray transport device further includes a label identification unit configured to scan label information provided on the specimen collection tool.

In addition, the tray transport device transmits label information identified by the label identification unit to the management server, and outputs information for confirming whether a specimen collection tool corresponding to the label information is a specimen collection tool matched to the input patient information.

The tray transport device transmits label information identified by the label identification unit to the management server, and the management server records information indicating that specimen collection corresponding to the label information has been performed among specimen collections required for the input patient.

In addition, the output unit includes a display unit configured to display required specimen collection details for the input patient.

In addition, the display unit outputs the number and types of specimen collections to be performed for the input patient.

In addition, the display unit outputs specimen collections that have been performed among the number and types of specimen collections to be performed for the input patient. In this case, the performed specimen collection is specimen collection corresponding to the identified label information.

### Effect

The method and system for automating patient-specific specimen collection according to the present disclosure can easily classify specimen collection tools required for respective patients, thereby reducing the time required to prepare and classify specimen collection tools for respective patients.

In addition, the method and system for automating patient-specific specimen collection according to the present disclosure can prevent medical errors by automatically confirming whether specimen collection tools are matched to respective patients.

In addition, the method and system for automating patient-specific specimen collection according to the present disclosure can prevent omissions in specimen collection by centrally managing records of completed specimen collections among specimen information required for respective patients.

### BRIEF DESCRIPTION OF DRAWINGS

FIG. 1 is a block diagram illustrating a configuration of a patient-specific specimen collection automation system according to an embodiment of the present disclosure.

FIG. 2 is a flowchart for describing a patient-specific specimen collection automation method according to an embodiment of the present disclosure.

FIG. 3 is a perspective view of a tray supply device according to an embodiment of the present disclosure.

FIG. 4 is an exploded view of the tray supply device according to an embodiment of the present disclosure.

FIG. 5 is a view for describing a blood collection tube supply unit included in the tray supply device according to an embodiment of the present disclosure.

FIG. 6 is a partially enlarged view of the blood collection tube supply unit according to an embodiment of the present disclosure.

FIG. 7 is a view for describing a label sheet supply unit included in the tray supply device according to an embodiment of the present disclosure.

FIG. 8 is a view for describing a tray supply unit according to an embodiment of the present disclosure.

FIG. 9 is a partially enlarged view for describing a tray moving unit of the tray supply unit according to an embodiment of the present disclosure.

FIG. 10 is a partially enlarged view for describing a first tray loading unit of the tray supply unit according to an embodiment of the present disclosure.

FIG. 11 is a partially enlarged view for describing a second tray loading unit of the tray supply unit according to an embodiment of the present disclosure.

FIG. 12 is a perspective view of a tray transport device according to an embodiment of the present disclosure.

### DETAILED DESCRIPTION OF THE EMBODIMENTS

Hereinafter, embodiments of the present disclosure will be described in detail with reference to the accompanying drawings so that those of ordinary skill in the art to which the present disclosure pertains can easily practice the embodiments.

However, the present disclosure may be implemented in various different forms and is not limited to the embodiments described herein. In the drawings, portions unrelated to the description are omitted in order to clearly describe the present disclosure, and like reference numerals are assigned to like portions throughout the present specification.

In the present specification, redundant descriptions of the same components will be omitted.

In addition, in the present specification, when a component is referred to as being “connected” or “coupled” to another component, it should be understood that the component may be directly connected or coupled to the other component, or that another component may be interposed therebetween. On the other hand, in the present specification, when a component is referred to as being “directly connected” or “directly coupled” to another component, it should be understood that no other component is interposed therebetween.

In addition, terms used in the present specification are used merely to describe specific embodiments and are not intended to limit the present disclosure.

In addition, in the present specification, singular expressions may include plural expressions unless the context clearly indicates otherwise.

In addition, in the present specification, terms such as “comprise”, “include” or “have” are merely intended to indicate the presence of features, numbers, steps, operations, components, parts, or combinations thereof described in the present specification, and should be understood as not precluding in advance the presence or addition of one or more other features, numbers, steps, operations, components, parts, or combinations thereof.

In addition, in the present specification, the term “and/or” includes any combination of a plurality of listed items or any one of the plurality of listed items. In the present specification, “A and/or B” may include “A,” “B,” or “both A and B.”

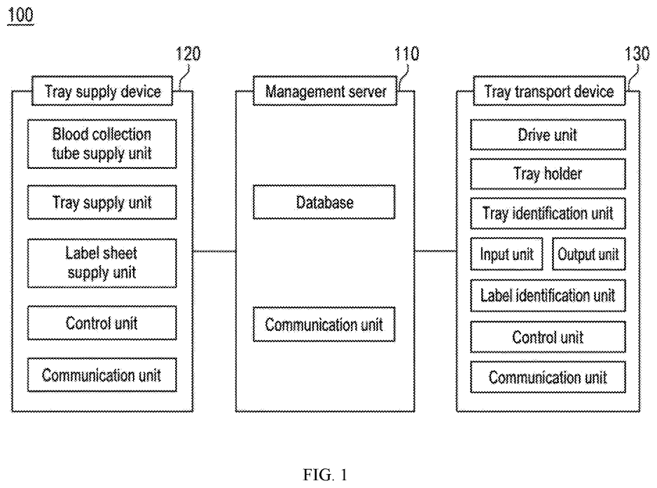

FIG. 1 is a block diagram illustrating a configuration of a patient-specific specimen collection automation system according to an embodiment of the present disclosure.

The patient-specific specimen collection automation system (100) according to the present disclosure automatically prepares and classifies specimen collection tools corresponding to specimen information required for respective patients in a hospital. In addition, the patient-specific specimen collection automation system (100) according to the present disclosure automatically records the progress of specimen collection. To this end, the patient-specific specimen collection automation system (100) according to the present disclosure includes a management server (110), a tray supply device (120), and a tray transport device (130).

The management server (110) may be an in-hospital server including specimen information required for respective patients. The management server (110) may include a database configured to store data and a communication unit configured to communicate with the tray supply device (120) and the tray transport device (130).

The database of the management server (110) includes patient information and specimen information required for respective patients. In addition, the database may include information on specimen collections performed for respective patients. In addition, the database may include identification information of trays matched to respective patients by the tray supply device (120).

Patient information and specimen information required for respective patients are input by an in-hospital administrator.

Information on specimen collections performed for respective patients may be input from the tray transport device (130) through a communication unit (not shown).

Identification information of trays matched to respective patients may be input from the tray supply device (120) through the communication unit (not shown). Here, each of the trays matched to respective patients includes specimen collection tools required for the corresponding patient.

In some cases, the management server (110) may be a component included in the tray supply device (120).

The tray supply device (120) is a device configured to distribute and supply specimen collection tools required for respective patients to an identified tray. The tray supply device includes a blood collection tube supply unit, a tray supply unit, a communication unit, and a control unit. Additionally, the tray supply device may include a label sheet supply unit.

The blood collection tube supply unit labels introduced empty blood collection tubes and supplies the labeled empty blood collection tubes to the identified tray. Information labeled on an empty blood collection tube may be information corresponding to specimen information required for respective patients. The specimen information required for respective patients may be received from the management server (110) through the communication unit.

The tray supply unit classifies specimen collection tools and label sheets required for respective patients into a tray including identification information. Specifically, the tray supply unit classifies blood collection tubes labeled for respective patients and label sheets attachable to specimen collection tools into the identified tray. Tray identification information matched to respective patients is transmitted to the management server (110). In some cases, the tray identification information may be transmitted to the tray transport device (130).

The tray supply unit may include a tray moving unit configured to move a tray, and a tray identification unit configured to confirm identification information indicated on the tray.

The label sheet supply unit supplies label sheets attachable to specimen collection tools other than blood collection tubes. Label sheets may be attached to various specimen collection tools, such as paper cups, urine containers, and stool containers, and may include information on specimen types for respective patients. In some cases, a label sheet may be used to record not only information on a purpose of specimen collection but also information related to medication to be taken by a corresponding patient. Here, the information related to medication may include at least one of a type of medication to be taken by the corresponding patient, an amount of the medication, a method of taking the medication, and information necessary to receive the medication.

The tray includes unique identification information, and the tray identification unit confirms the unique identification information. The tray supply device (120) identifies a tray and classifies all of the blood collection tubes and label sheets required for a specific patient by placing them in the corresponding tray. For example, the specific patient may require three blood collection tubes and one urine container for specimen collection. In this case, the tray supply device (120) classifies three blood collection tubes for the specific patient and a label sheet attachable to the urine container by placing them in the identified tray.

The communication unit provided in the tray supply device (120) (hereinafter, referred to as a supply device communication unit) communicates with the management server (110). In some cases, the supply device communication unit communicates with the tray transport device (130). The tray supply device (120) may receive specimen information required for respective patients from the management server (110) through the supply device communication unit, and may transmit, in response thereto, identification information of trays classified for respective patients to the management server (110).

The control unit of the tray supply device (120) controls the blood collection tube supply unit, the tray supply unit, the label sheet supply unit, and the supply device communication unit.

The tray transport device (130) is a device configured to transport trays into which specimen collection tools and label sheets required for respective patients have been classified by the tray supply device (120). The tray transport device (130) includes a drive unit, a tray holder, a tray identification unit, a communication unit, an output unit, and a control unit. Additionally, the tray transport device (130) may further include at least one of an input unit and a label identification unit.

The drive unit of the tray transport device (130) may be driven in a manual or electric manner. The drive unit may be provided in a body of the tray transport device (130). The body of the tray transport device (130) includes a storage space, and various specimen collection tools, such as empty blood collection tubes, paper cups, urine containers, and stool containers, may be provided therein.

The tray holder may receive a tray supplied from the tray supply device (120). The tray holder may include partitioned spaces so that a tray can be received in each of the spaces.

The tray identification unit (430) is provided in the tray holder and confirms identification information of the tray. The tray identification unit (430) may be provided in each partitioned space of the tray holder.

The communication unit of the tray transport device (130) (hereinafter, referred to as a transport device communication unit) may receive specimen information required for respective patients and tray identification information matched to respective patients from the management server (110). In some cases, tray identification information matched to respective patients may be received through the tray supply device (120).

The output unit of the tray transport device (130) may indicate a corresponding tray in response to patient information. The output unit may indicate, in the tray holder, a partitioned space in which a tray matched to the patient information is received.

The input unit of the tray transport device (130) may be used to input patient information.

The label identification unit of the tray transport device (130) may be used to confirm label information of blood collection tubes and label sheets included in the tray. The confirmed label information may be transmitted to the management server (110) through the transport device communication unit and recorded.

The control unit of the tray transport device (130) may control the tray identification unit, the output unit, and the communication unit. When the drive unit is automatic, the control unit may control the drive unit, and may control the input unit and the label identification unit provided in the tray transport device (130).

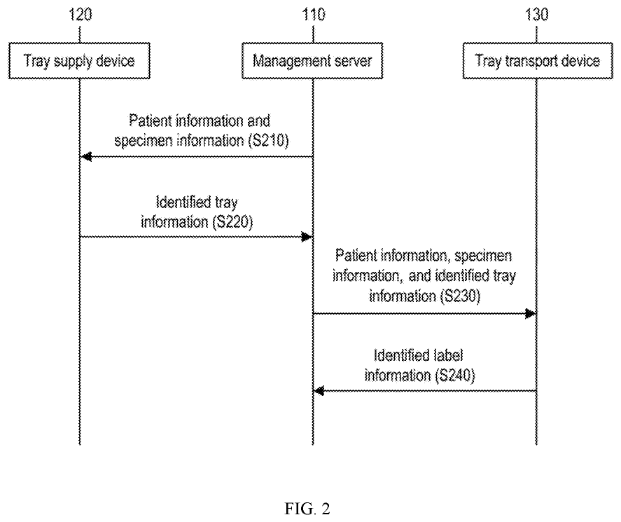

FIG. 2 is a flowchart for describing a patient-specific specimen collection automation method according to an embodiment of the present disclosure. Specifically, FIG. 2 is a view for describing a method for automating patient-specific specimen collection using the patient-specific specimen collection automation system of FIG. 1.

In the patient-specific specimen collection automation method according to the present disclosure, specimen information required for respective patients is first input. Blood collection tubes labeled to correspond to the input specimen information are classified into an identified tray. Thereafter, the tray receiving the labeled blood collection tubes is transported to a location near the respective patient. Thereafter, trays matched to respective patients are identified.

The inputting of the specimen information required for respective patients is a step in which patient information and the specimen information required for respective patients are input to the management server (110) by hospital personnel. The patient information and the specimen information input to the management server (110) may be transmitted to the tray supply device (120). (S210)

The classifying of the labeled blood collection tubes into the identified tray is a step performed by the tray supply device (120). This may be a step of preparing blood collection tubes labeled to correspond to specimen information for respective patients and label sheets, and classifying the prepared blood collection tubes and label sheets into trays matched to respective patients. Here, tray identification information matched to respective patients may be transmitted to the management server (110). (S220)

The classifying of the labeled blood collection tubes into the identified tray may further include classifying, into the identified tray, a label sheet corresponding to the specimen information received from the management server (110). That is, the classifying may be a step of classifying the labeled blood collection tubes and label sheets usable for specimen collection tools other than blood collection tubes into the identified tray for a respective patient.

The management server (110) may record tray identification information matched to respective patients and transmit the tray identification information to the tray transport device (130). Specifically, the management server (110) may transmit patient information, specimen information, and tray identification information to the tray transport device (130). In some cases, tray identification information matched to respective patients may be directly transmitted from the tray supply device (120) to the tray transport device (130).

The transporting of the tray is a step of transporting the tray from the tray supply device (120) to the respective patient, manually or automatically, using the tray transport device (130).

The identifying of the trays matched to respective patients is a step of identifying a tray including specimen collection tools and label sheets in response to patient information input to the tray transport device (130). When hospital personnel input patient information to the tray transport device (130), the tray transport device (130) identifies and indicates a tray matched to the input patient information using information received through the management server (110).

After identifying the tray, the method may further include identifying label information of specimen collection tools included in the tray. The identifying of the label information includes confirming label information of a blood collection tube. In addition, the identifying of the label information includes confirming label information of a label sheet.

For example, when three blood collection tubes and one label sheet for a urine container are included in a tray for a specific patient, the identifying of the label information is a step of confirming four pieces of label information.

The identifying of the label information may further include confirming whether the identified label information matches input patient information, and inputting information indicating that specimen collection corresponding to the identified label information has been completed. The confirming of whether the identified label information matches the input patient information is a step of reconfirming whether label information of a specimen collection tool included in the tray matches a patient. When the identified label information does not match the input patient, matching between identification information of the tray and the patient is incorrect. By providing notification thereof, incorrect specimen collection is prevented. When the identified label information matches the input patient, information indicating that specimen collection corresponding to the identified label information has been completed is input.

For example, a case in which three blood collection tubes and one label sheet for a urine container are included in a tray for an input patient will be described. Hospital personnel take one blood collection tube from the tray and identify label information. When the label information matches the input patient, information indicating that specimen collection corresponding to the identified label information has been completed among four specimen collections may be input in the identifying of the label information.

The identified label information may be transmitted to the management server (110). (S240) The management server (110) may collectively manage omitted specimen collections and completed specimen collections for respective patients.

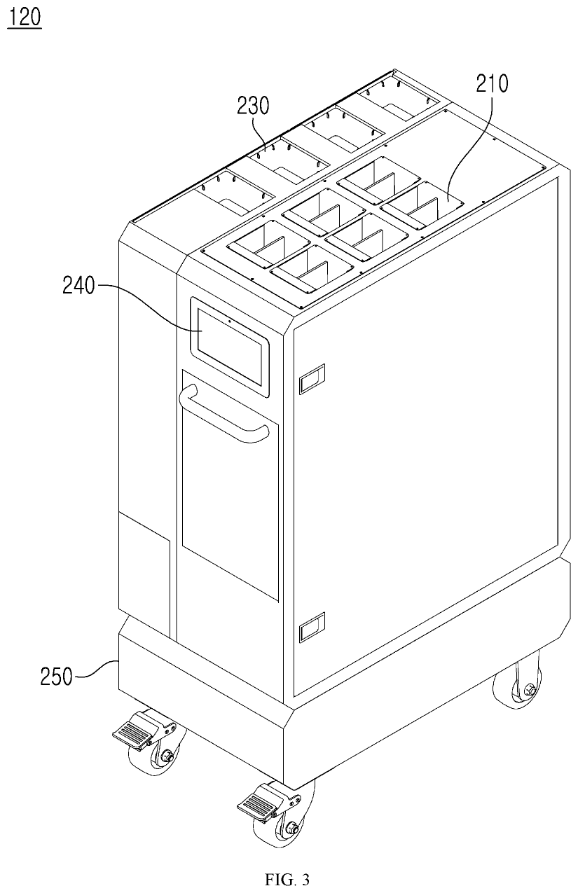

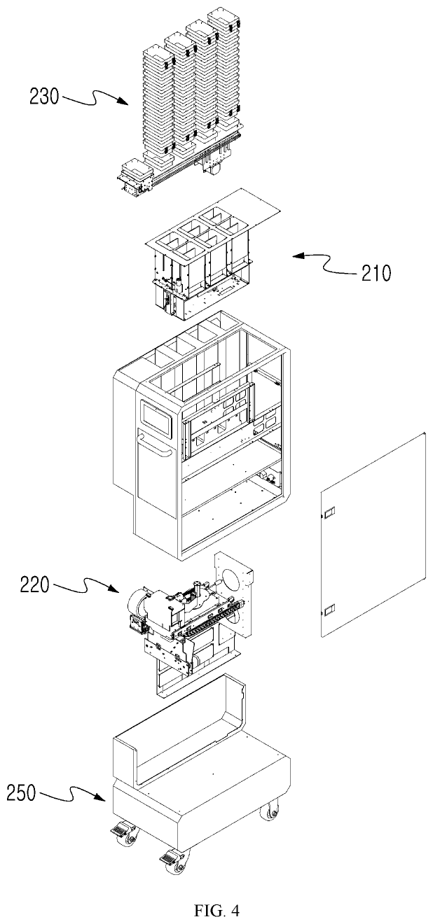

FIG. 3 is a perspective view of a tray supply device according to an embodiment of the present disclosure. FIG. 4 is an exploded view of the tray supply device according to an embodiment of the present disclosure. Specifically, FIGS. 3 and 4 are views for describing the tray supply device (120) included in the patient-specific specimen collection automation system of FIG. 1.

The tray supply device (120) includes a blood collection tube supply unit (210), a tray supply unit (230), and a supply device communication unit (not shown). In addition, the tray supply device (120) may further include a label sheet supply unit (220). In addition, the tray supply device may further include an input and control display (240) and a driving unit (250).

When empty blood collection tubes are introduced, the blood collection tube supply unit (210) labels the empty blood collection tubes to correspond to specimen information required for respective patients and then discharges the labeled empty blood collection tubes.

The tray supply unit (230) is a device configured to classify blood collection tubes supplied from the blood collection tube supply unit (210) into trays for respective patients.

The supply device communication unit is a device configured to communicate with the management server (110) in order to receive specimen information required for respective patients. A tray includes unique identification information, and the identification information of the tray and patient information may be matched and transmitted to the management server (110). Identification information of trays matched to respective patients may be transmitted to the management server (110) through the supply device communication unit.

The label sheet supply unit (220) is a device configured to supply label sheets corresponding to specimen information required for respective patients.

The input and control display (240) may be a display used to control the tray supply device (120) and to display a progress status of the tray supply device. In addition, the input and control display (240) may be a display used to input additional specimen information for respective patients other than information received through the management server (110).

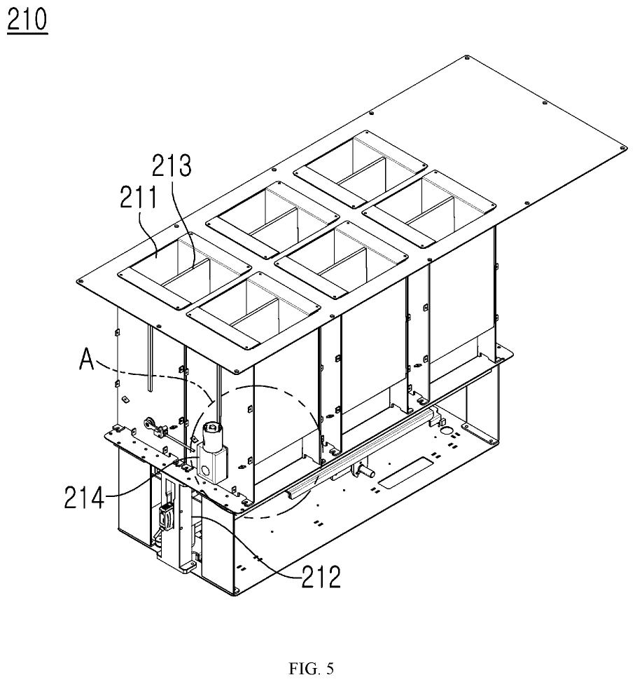

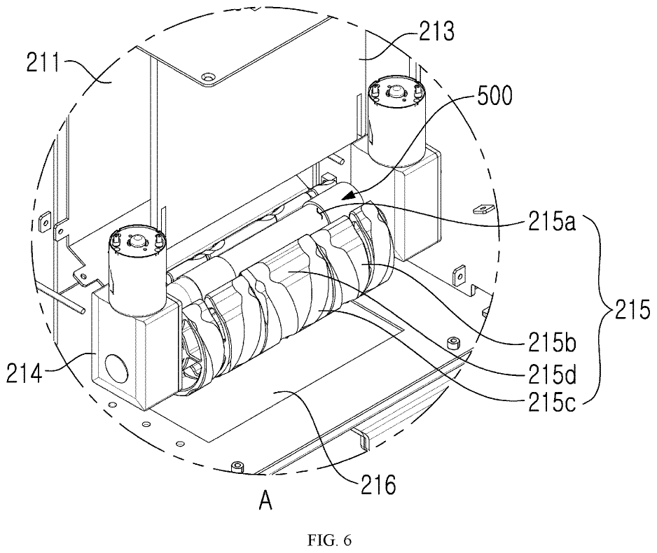

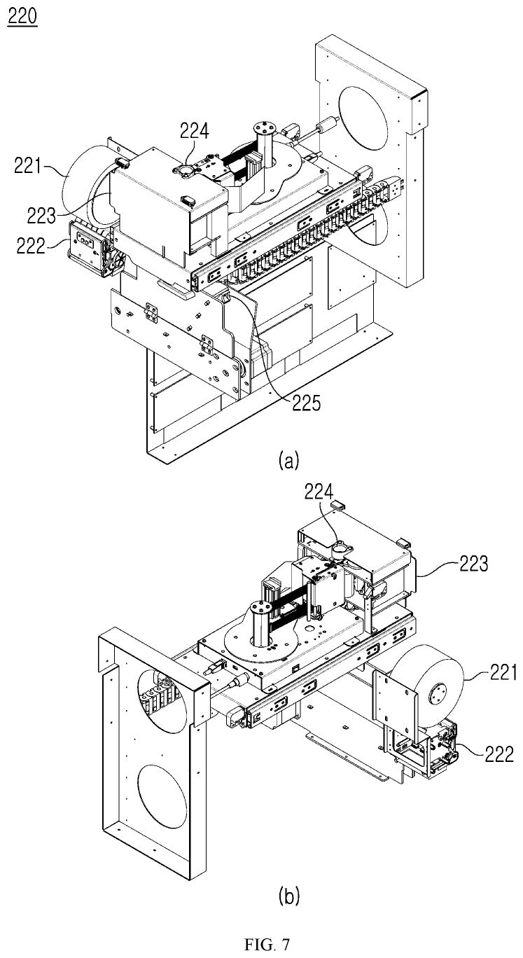

FIG. 5 is a view for describing a blood collection tube supply unit included in a tray supply device according to an embodiment of the present disclosure. FIG. 6 is a partially enlarged view of the blood collection tube supply unit according to an embodiment of the present disclosure. FIG. 7 is a view for describing a label sheet supply unit included in the tray supply device according to an embodiment of the present disclosure.

The blood collection tube supply unit (210) according to an embodiment of the present disclosure is a device into which empty blood collection tubes are introduced and which labels and supplies the empty blood collection tubes. The blood collection tube supply unit (210) needs to have a function of separating and aligning randomly introduced empty blood collection tubes one by one. To this end, the blood collection tube supply unit (210) includes a blood collection tube inlet hole (212), an inlet hole partition (213), a roller unit (215), and a blood collection tube discharge hole (216).

The blood collection tube inlet hole (212) has a rectangular shape and is formed such that one side thereof is longer than a length of a blood collection tube.

The inlet hole partition (213) may be a partition configured to partition the blood collection tube inlet hole along one direction. The inlet hole partition (213) may prevent a blood collection tube from being introduced into the blood collection tube inlet hole (212) in a direction perpendicular to the one direction on a horizontal plane.

The roller unit (215) is provided below the blood collection tube inlet hole (212) and performs a function of discharging randomly introduced blood collection tubes one by one to the blood collection tube discharge hole. Blood collection tubes introduced into the blood collection tube inlet hole (212) are guided to and collected at the roller unit (215). The roller unit (215) includes a roller unit main groove (215b) into which a blood collection tube can be introduced, and performs a function of discharging blood collection tubes one by one by being rotated by a roller unit driving motor (214).

The roller unit (215) may be arranged along a direction in which the inlet hole partition (213) is arranged, hereinafter referred to as the one direction, and the roller unit main groove (215b) may also be arranged in the one direction. The inlet hole partition (213) may prevent a blood collection tube from being introduced in a direction perpendicular to the one direction on a horizontal plane. However, the inlet hole partition (213) cannot prevent a blood collection tube from being vertically introduced.

When a blood collection tube is vertically introduced into the blood collection tube inlet hole (212), an end portion of the blood collection tube may be caught in the roller unit main groove (215b), causing a problem in which the roller unit (215) cannot rotate. To prevent this, it is necessary to lay the blood collection tube introduced vertically along the one direction.

To this end, the roller unit (215) may include a roller unit body (215a), the roller unit main groove (215b), and a roller unit sub-groove (215c). Additionally, the roller unit (215) may further include a blood collection tube catching groove (215d).

The roller unit body (215a) is provided along the one direction. The roller unit driving motor (214) rotates the roller unit body (215a).

The roller unit main groove (215b) is a groove provided in a side surface of the roller unit body (215a) along the one direction. The roller unit main groove (215b) has a size sufficient for a blood collection tube to be introduced therein.

The roller unit sub-groove (215c) is a groove recessed obliquely around a side surface of the roller unit body (215a). When an end portion of the blood collection tube introduced vertically is caught in the roller unit sub-groove (215c), the blood collection tube may be laid along the one direction by rotationally driving the roller unit body (215a).

The roller unit sub-groove (215c) may be recessed obliquely around the side surface of the roller unit body (215a) such that the roller unit sub-groove (215c) is connected to one side and the other side of the roller unit main groove (215b).

The blood collection tube catching groove (215d) may be a groove provided adjacent to the roller unit main groove (215b) along the one direction. The blood collection tube catching groove (215d) is a groove configured to prevent an end portion of a blood collection tube from being introduced into the roller unit main groove (215b).

The blood collection tube discharge hole (216) is a hole through which blood collection tubes discharged one by one by rotation of the roller unit (215) are discharged.

Blood collection tubes discharged through the blood collection tube discharge hole (216) may be vertically arranged, labeled by a blood collection tube labeling unit (223), and then supplied to a tray. A blood collection tube vertical fixing unit (224) vertically supports a blood collection tube while the blood collection tube is being labeled. A blood collection tube for which labeling has been completed may be supplied to the tray through a blood collection tube discharge guide unit (225).

The label sheet supply unit (220) according to the present disclosure includes a label sheet supply roll (221) and a label sheet printing unit (222).

The label sheet supply unit (220) supplies label sheets that are not used for labeling blood collection tubes but are attachable to other specimen collection tools.

The label sheet printing unit (222) prints specimen information for respective patients on label sheets supplied from the label sheet supply unit (220). The printed label sheets may be supplied to the tray.

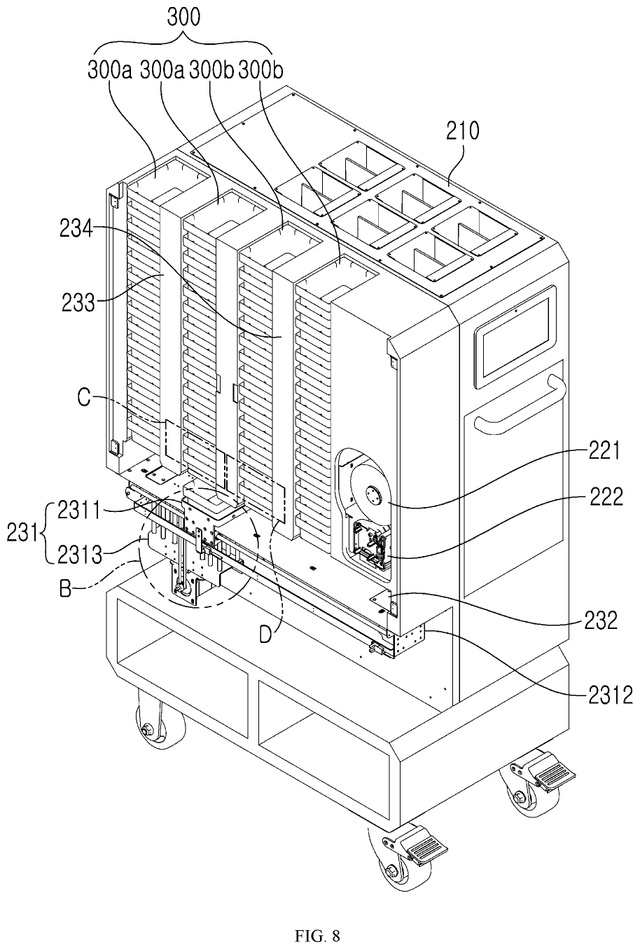

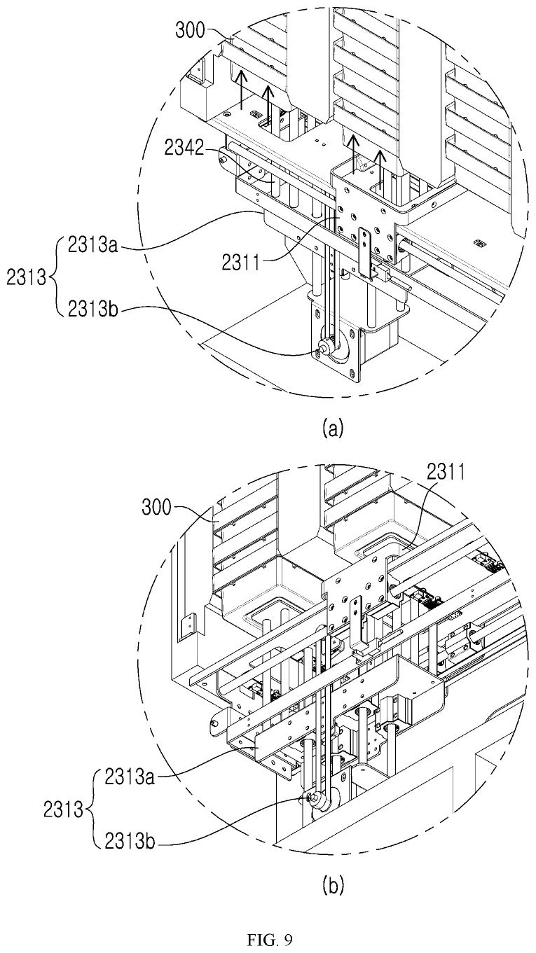

FIG. 8 is a view for describing a tray supply unit according to an embodiment of the present disclosure. FIG. 9 is a partially enlarged view for describing a tray moving unit of the tray supply unit according to an embodiment of the present disclosure. Specifically, FIG. 9 is a partially enlarged view of region B of FIG. 8.

The tray supply unit (230) according to an embodiment of the present disclosure includes a tray moving unit (231) and a tray identification unit (232).

The tray moving unit (231) is a component configured to move a tray. The tray moving unit (231) is a component configured to move an empty tray (300b) to a location where blood collection tubes and label sheets are supplied, and then move a filled tray (300a) to another location.

The tray identification unit (232) is a component configured to confirm unique identification information provided on a tray. Tray identification information may be provided on a lower portion of the tray and may be identified by the tray identification unit (232) through near-field wireless communication.

The tray identification unit (232) may be provided at a position where blood collection tubes and label sheets are supplied. The tray moving unit (231) moves an empty tray to the tray identification unit (232), and identification information of the tray is confirmed. After blood collection tubes and label sheets for a specific patient are supplied to the identified tray, tray identification information and information of the specific patient are matched. Tray identification information matched to respective patients is transmitted to the management server (110).

The tray supply unit (230) may further include a first tray loading unit (233) and a second tray loading unit (234). Filled trays (300a) each filled with blood collection tubes for a respective patient may be stacked in the first tray loading unit (233). Empty trays may be stacked in the second tray loading unit (234).

The tray moving unit (231) moves an empty tray (300b) supplied from the second tray loading unit (234) to the tray identification unit (232) so that the empty tray (300b) is identified. The tray moving unit (231) moves a filled tray (300a) containing blood collection tubes and label sheets, and stacks the filled tray (300a) in the first tray loading unit (233).

The tray moving unit (231) includes a tray guide (2311), a guide driving unit (2312), and a vertical driving unit (2313).

The tray guide (2311) is a component configured to surround side surfaces of the tray. Since tray identification information is provided at a lower portion of the tray, the tray guide (2311) does not surround the lower portion of the tray.

The guide driving unit (2312) is a driving unit configured to move the tray guide (2311). The guide driving unit (2312) moves the tray guide (2311) surrounding the tray so that the tray can be positioned at the first tray loading unit (233), the second tray loading unit (234), and the tray identification unit (232).

The vertical driving unit (2313) is a component provided below the first tray loading unit (233) and configured to vertically move the tray. Filled trays containing blood collection tubes and label sheets for respective patients may be stacked in the first tray loading unit (233) by the vertical driving unit.

Specifically, the vertical driving unit (2313) is positioned below the first tray loading unit (233), and includes a vertical moving component (2313a) configured to move vertically and a vertical driving motor (2313b) configured to vertically drive the vertical moving component (2313a).

A filled tray is stacked in the first tray loading unit (233) as the vertical driving unit (2313) moves upward while the filled tray is positioned below the first tray loading unit (233).

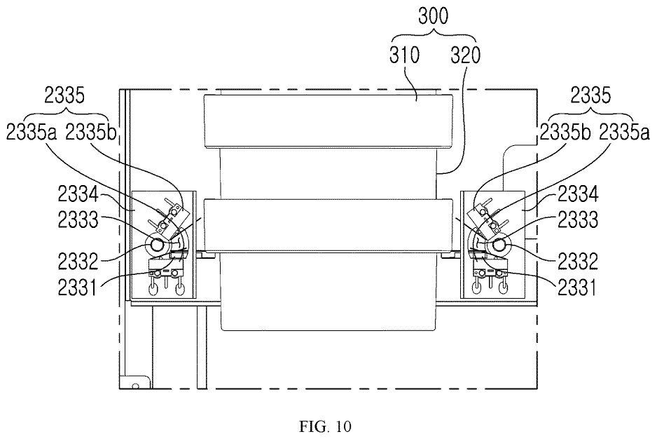

FIG. 10 is a partially enlarged view for describing the first tray loading unit of the tray supply unit according to an embodiment of the present disclosure. Specifically, FIG. 10 is an enlarged view of region C of FIG. 8.

The first tray loading unit (233) according to an embodiment of the present disclosure is a component in which filled trays containing blood collection tubes, label sheets, and the like are stacked. A filled tray is introduced from below the first tray loading unit (233) and stacked.

A tray can be introduced into a lower portion of the first tray loading unit (233) without restriction, but should be prevented from falling out due to gravity. To this end, it is necessary to define a particular structure of the tray. The tray (300) may have a stepped structure defining an upper portion and a lower portion, and may be formed such that an upper perimeter (310) is larger than a lower perimeter (320).

The first tray loading unit (233) includes a first support portion (2331), a first guide groove (2333), and a first sensor unit (2335).

The first support portion (2331) may be provided on both lateral sides of a space in which trays are stacked. The first support portion is rotatably connected to a rotation shaft (2332) and supports the tray by engaging with the stepped structure of the tray.

The first guide groove (2333) limits a rotation angle of the first support portion. Specifically, a protrusion of the first support portion (2331) is inserted into the first guide groove (2333), so that the rotation angle is limited. The first guide groove (2333) may have an arc shape that allows the first support portion (2331) to rotate upward by a predetermined angle from a horizontal state.

The first sensor unit (2335) may be a component configured to detect rotation of the first support portion (2331). When the first support portion (2331) rotates sufficiently to allow a tray to be inserted, the first sensor unit (2335) may detect the rotation and thus detect that a tray is additionally stacked.

To this end, the first sensor unit (2335) may include a lower first sensor unit (2335a) and an upper first sensor unit (2335b). When the first support portion (2331) rotates and the lower first sensor unit (2335a) and the upper first sensor unit (2335b) come into contact with each other, the first sensor unit (2335) may detect that a tray is additionally stacked.

Additionally, the first tray loading unit (233) may include sidewalls (2334) supporting the rotation shaft (2332) on both lateral sides of the space in which trays are stacked.

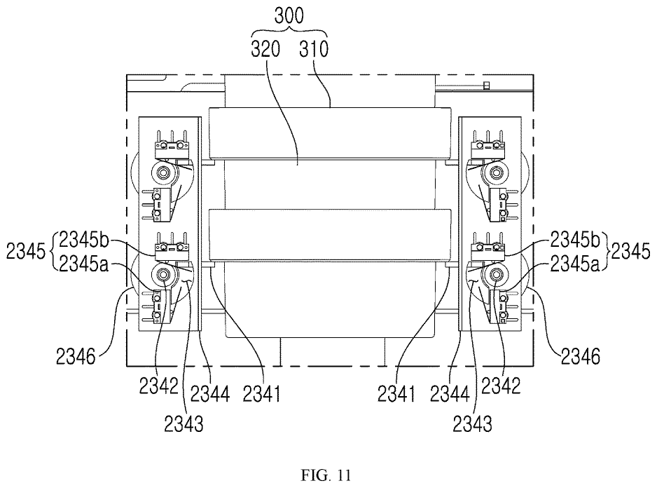

FIG. 11 is a partially enlarged view for describing the second tray loading unit of the tray supply unit according to an embodiment of the present disclosure. Specifically, FIG. 11 is an enlarged view of region D of FIG. 8.

The second tray loading unit (234) according to an embodiment of the present disclosure is a component in which empty trays are stacked. An empty tray may be withdrawn downward and moved by the tray moving unit.

Trays withdrawn from a lower portion of the second tray loading unit (234) should be withdrawn one by one. To this end, it is necessary to define a particular structure of the tray. The tray (300) may have a stepped structure defining an upper portion and a lower portion, and may be formed such that an upper perimeter (310) is larger than a lower perimeter (320).

The second tray loading unit (234) includes a second support portion (2341), a rotation motor (2346), and a second sensor unit (2345).

The second support portion (2341) may be provided on both lateral sides of a space in which trays are stacked. The second support portion is rotatably connected to a rotation shaft (2342) and supports the tray by engaging with the stepped structure of the tray.

The rotation motor (2346) may be a component connected to the rotation shaft (2342) and configured to rotationally drive the second support portion (2341).

The second guide groove (2343) limits a rotation angle of the second support portion. Specifically, a protrusion of the second support portion (2341) is inserted into the second guide groove (2343), so that the rotation angle is limited. The second guide groove (2343) may have an arc shape that allows the second support portion (2341) to rotate downward by a predetermined angle from a horizontal state.

The second sensor unit (2345) may be a component configured to detect rotation of the second support portion (2341). When the second support portion (2341) rotates sufficiently to allow a tray to be withdrawn, the second sensor unit (2345) may detect the rotation and thus detect that the tray is withdrawn.

To this end, the second sensor unit (2345) may include a lower second sensor unit (2345a) and an upper second sensor unit (2345b). When the second support portion (2341) rotates and the lower second sensor unit (2345a) and the upper second sensor unit (2345b) come into contact with each other, the second sensor unit (2345) may detect that the tray is withdrawn.

In the second tray loading unit (234), trays should be withdrawn one by one. To this end, the second tray loading unit (234) preferably includes two sets arranged vertically, each set including the second support portion (2341), the rotation motor (2346), and the second sensor unit (2345).

First, the second support portion (2341) provided at an upper position is fixed in a horizontal state and supports an empty tray located on a second layer. In this state, the second support portion (2341) provided at a lower position is opened to supply an empty tray located on a first layer to the tray moving unit.

Thereafter, the second support portion (2341) provided at a lower position is fixed in a horizontal state to prevent an empty tray from being supplied to the tray moving unit. In this state, the second support portion (2341) provided at an upper position is opened to allow the stacked empty trays to move downward by one layer.

The second tray loading unit (234) may include sidewalls (2344) supporting the rotation shaft (2342) on both lateral sides of the space in which trays are stacked.

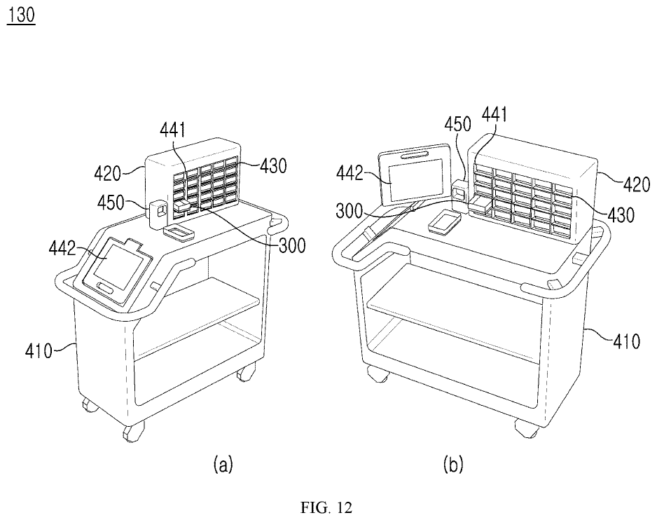

FIG. 12 is a perspective view of a tray transport device according to an embodiment of the present disclosure. Specifically, FIG. 12 is a view for describing the tray transport device (130) included in the patient-specific specimen collection automation system of FIG. 1.

The tray transport device (130) according to an embodiment of the present disclosure includes a drive unit (410), a tray holder (420), a tray identification unit (430), a transport device communication unit (not shown), an input unit (442), and an output unit (440).

The tray transport device (130) is a device configured to transport trays into which specimen collection tools and label sheets required for respective patients have been classified by the tray supply device (120).

The drive unit (410) of the tray transport device (130) may be driven in a manual or electric manner. The drive unit may be provided in a body of the tray transport device (130). The body of the tray transport device (130) includes a storage space, and various specimen collection tools, such as empty blood collection tubes, paper cups, urine containers, and stool containers, may be provided therein.

A tray supplied from the tray supply device (120) may be received in the tray holder. The tray holder may include partitioned spaces, each configured to receive a tray. The partitioned spaces may be provided in a matrix form in rows and columns.

The tray identification unit (430) is provided in the tray holder and confirms identification information of the tray. The tray identification unit (430) may be provided in each partitioned space of the tray holder.

The communication unit of the tray transport device (130), hereinafter referred to as a transport device communication unit, may receive specimen information required for respective patients and tray identification information matched to respective patients from the management server (110). In some cases, tray identification information matched to respective patients may be received through the tray supply device (120).

The output unit (440) of the tray transport device (130) may indicate a corresponding tray in response to patient information. The output unit (440) may indicate, in the tray holder, a partitioned space in which a tray matched to the patient information is received.

The output unit (440) may be an LED provided in each partitioned space of the tray holder (420) and configured to indicate a section in which a tray matched to the input patient information is received.

The output unit (440) may further include a display unit configured to display specimen collection details required for the input patient. In this case, the display unit may perform a function of the input unit (442).

The input unit (442) of the tray transport device (130) may be used to input patient information.

The control unit of the tray transport device (130) may control the tray identification unit, the output unit, and the communication unit. When the drive unit (410) is automatic, the control unit may control the drive unit, and may control the input unit and the label identification unit provided in the tray transport device (130).

The label identification unit (450) of the tray transport device (130) may be used to confirm label information of blood collection tubes and label sheets included in the tray. The confirmed label information may be transmitted to the management server (110) through the transport device communication unit and recorded.

Label information identified by the label identification unit (450) may be transmitted to the management server, and information for confirming whether a specimen collection tool corresponding to the label information is matched to the input patient information may be received.

When the label information identified by the label identification unit (450) and the input patient information do not match, a specimen is not collected using the specimen collection tool corresponding to the identified label information. This may occur when matching between tray identification information and the patient has been incorrectly input, or when another specimen collection tool has been introduced.

When the label information identified by the label identification unit (450) and the input patient information match, information indicating that, among the specimen collections required for the input patient, specimen collection corresponding to the identified label information has been performed may be input to the management server.

The display unit may output the number and types of specimen collections to be performed for the input patient. In addition, the display unit may output information on specimen collections performed among the number and types of specimen collections to be performed for the input patient. In this case, the performed specimen collection may be specimen collection corresponding to the identified label information.

Although exemplary embodiments of the present disclosure have been described in detail above, those of ordinary skill in the art to which the present disclosure pertains will understand that various modifications may be made to the above-described embodiments without departing from the scope of the present disclosure. Therefore, the scope of the present disclosure should not be determined as being limited to the described embodiments, and should be determined by the claims described below and equivalents of the claims.

What is claimed is:

1. A patient-specific specimen collection automation system comprising:
a management server including specimen information required for respective patients;
a tray supply device configured to supply, to a tray, blood collection tubes labeled to correspond to the specimen information; and
a tray transport device configured to transport the tray,
wherein the tray supply device includes:
a supply device communication unit configured to communicate with the management server;
a blood collection tube supply unit configured to supply the blood collection tubes labeled to correspond to the specimen information; and
a tray supply unit configured to classify the labeled blood collection tubes for respective patients into the tray, the tray including identification information, and
wherein the tray transport device includes:
a transport device communication unit configured to communicate with the management server;
a tray holder configured to receive the tray supplied from the tray supply device;
a tray identification unit configured to obtain the identification information of the tray; and
an output unit configured to indicate trays matched to respective patients.

2. The patient-specific specimen collection automation system according to claim 1,
wherein the tray supply unit includes:
a tray moving unit configured to move the tray; and
a tray identification unit configured to confirm the identification information indicated on the tray.

3. The patient-specific specimen collection automation system according to claim 2,
wherein the tray supply device further includes a label sheet supply unit configured to supply, to the tray, a label sheet corresponding to the specimen information.

4. The patient-specific specimen collection automation system according to claim 3,
wherein the tray supply device is configured to classify at least one of the blood collection tubes labeled for respective patients and the label sheet into the tray identified by the tray identification unit, and transmit identified tray information for respective patients to the management server.

5. The patient-specific specimen collection automation system according to claim 1,
wherein the tray transport device further includes a label identification unit configured to identify label information.

6. The patient-specific specimen collection automation system according to claim 5,
wherein the tray transport device is configured to identify label information of a specimen collection tool included in the tray identified by the tray identification unit, and transmit, to the management server, specimen collection completion information for respective patients corresponding to the identified label information.

7. A patient-specific specimen collection automation method comprising:
inputting specimen information required for respective patients;
classifying blood collection tubes labeled to correspond to the specimen information into an identified tray;
transporting the identified tray; and
identifying trays matched to respective patients.

8. The patient-specific specimen collection automation method according to claim 7,
wherein the classifying of the labeled blood collection tubes further includes classifying a label sheet corresponding to the specimen information into the identified tray.

9. The patient-specific specimen collection automation method according to claim 8, further comprising:
identifying label information of a specimen collection tool included in the identified tray; and
inputting specimen collection completion information corresponding to the identified label information.

10. The patient-specific specimen collection automation method according to claim 9,
wherein identifying the label information further includes:
confirming whether the identified label information matches input patient information; and
inputting, when the identified label information matches the input patient information, the specimen collection completion information corresponding to the identified label information.
